(19) United States
(12) Patent Application Publication
(10) Pub. No.: US 2021/0346270 A1
(43) Pub. Date: Nov. 11, 2021

(54) WATER-BASED COSMETIC

(71) Applicant: TOKIWA CORPORATION, Gifu (JP)

(72) Inventor: Shunpei TAKEZAWA, Saitama (JP)

(73) Assignee: TOKIWA CORPORATION, Gifu (JP)

(21) Appl. No.: 17/226,575

(22) Filed: Apr. 9, 2021

(30) Foreign Application Priority Data
May 8, 2020 (JP) 2020-082705

(51) Int. Cl. A61K 8/81; A61K 8/73; A61K 8/02; A61K 8/26; A61K 8/891; A61Q 1/02

(52) U.S. Cl. CPC A61K 8/8147; A61K 8/731; A61K 8/0241; A61K 2800/41; A61K 8/891; A61Q 1/02; A61K 2800/546; A61K 8/26

(57) ABSTRACT

Provided is a water-based cosmetic that excels in adhesion, quick-drying property, spreadability without causing uneven application, and easiness of fine control of color density. A water-based cosmetic contains 2 to 18% by mass of a water-insoluble water absorbent polymer; 15 to 95% by mass of water; and 0.5 to 45% by mass of a powder, the water-based cosmetic having a penetration of 0.1 N or larger, where the penetration being hardness measured by using a pressure sensing shaft with a 10-mm-diameter spherical end, at a penetration speed of 6 cm/min, an insertion depth of 10 mm, and a temperature of 25° C.

### INCORPORATION BY REFERENCE

This application is based on Japanese Patent Application No. 2020-082705, filed on May 8, 2020, the content of which is incorporated hereinto by reference.

### BACKGROUND OF THE INVENTION

### 1. Field of the Invention

This invention relates to a water-based cosmetic.

### 2. Description of the Related Art

Makeup cosmetics such as eyebrow pencil, eyeliner, eye shadow, blush and foundation are required not only to provide good finish including adhesion and color development when applied on skin, but also to excel in quick-drying property, and easiness of fine control of color density.

Water-based cosmetics that contain water as a major ingredient have been widely used for makeup cosmetics, featured by their cool and refreshing touch of use. Popular types of them are water-based liquid cosmetics that exist in liquid form. The water-based liquid cosmetics however tend to cause settling or aggregation of colorant such as pigment, and tend to drip when applied. Thus, there has been proposed a technique of controlling viscosity with use of a water-soluble thickener.

More specifically, Patent Document 1 proposes a technique of blending clay mineral such as bentonite, or a water-soluble polysaccharides such as xanthane gum and carboxymethyl cellulose. Patent Document 2 discloses a cosmetic blended with an alcohol. Patent Document 3 proposes a water-based solid cosmetic obtainable with use of a solidifying agent such as agar.

### CITATION LIST

### Patent Document

[Patent Document 1] JP-2008-031057A

[Patent Document 2] JP-2002-161024A

[Patent Document 3] JP-2006-312610A

### SUMMARY OF THE INVENTION

A composition described in Patent Document 1, with increased viscosity ascribed to the water-soluble thickener, tends to degrade the quick-drying property and cause uneven application. On the other hand, enhanced drying speed attained by adding alcohol as described in Patent Document 2 makes it difficult to finely control the color density. Meanwhile, the solidified water-based cosmetic according to an aspect of Patent Document 3 tends to make, on the surface thereof, an applicator or skin more likely to slip, making it difficult to catch the cosmetic, making the cosmetic poorly adhesive and spreadable on skin, and making it difficult to finely control the density.

Considering the situations of the prior art, it is therefore an object of this invention to provide a water-based cosmetic that excels in adhesion, quick-drying property, spreadability without causing uneven application, and easiness of fine control of color density.

### Solution to Problem

<1> A water-based cosmetic comprising:

2 to 18% by mass of a water-insoluble water absorbent polymer;

15 to 95% by mass of water; and

0.5 to 45% by mass of a powder,

the water-based cosmetic having a penetration of 0.1 N or larger,

where the penetration being hardness measured by using a pressure sensing shaft with a 10-mm-diameter spherical end, at a penetration speed of 6 cm/min, an insertion depth of 10 mm, and a temperature of 25° C.

<2> The water-based cosmetic of <1>, wherein the water-insoluble water absorbent polymer has a swelling ratio of 5 to 2000 g/g.

<3> The water-based cosmetic of <1> or <2>, wherein the water-insoluble water absorbent polymer has a swollen particle size (D₅₀) of 1 μm or larger and 1000 μm or smaller.

<4> The water-based cosmetic of any one of <1> to <3>, wherein percentage of content (% by mass) of an aqueous medium, to a water absorption capacity given by a product of content (% by mass) of the water-insoluble water absorbent polymer and swelling ratio (g/g), is 10% or larger and smaller than 130% (mass basis).

<5> The water-based cosmetic of any one of <1> to <4>, wherein the water-insoluble water absorbent polymer contains at least one substance selected from crosslinked polyacrylate salt and cellulose derivative.

<6> The water-based cosmetic of any one of <1> to <5>, wherein content of the powder is 10 to 40% by mass.

<7> The water-based cosmetic of any one of <1> to <6>, wherein the powder contains a hydrophobic powder.

<8> The water-based cosmetic of any one of <1> to <7>, wherein the powder contains a coloring pigment.

<9> The water-based cosmetic of any one of <1> to <8>, further comprising a water-soluble dye.

<10> The water-based cosmetic of any one of <1> to <9>, wherein the penetration of the water-based cosmetic is from 0.3 N to 25 N.

<11> The water-based cosmetic of any one of <1> to <10>, wherein the water-insoluble water absorbent polymer contains at least one substance selected from crosslinked polyacrylate salt and cellulose derivative and the powder contains at least one kind of coloring pigment, pearl and extender pigment.

<12> The water-based cosmetic of any one of <1> to <11>, further comprising at least one kind of polyhydric alcohol, lower alcohol, water-soluble dye, water-soluble coating agent and viscosity modifier.

### Advantageous Effects of Invention

This invention succeeded in providing a water-based cosmetic that excels in adhesion, quick-drying property, spreadability without causing uneven application, and easiness of fine control of color density.

### BRIEF DESCRIPTION OF THE DRAWINGS

FIGS. 1(a) to 1(c) present photographs for explaining difference between insolubility and hydrophobicity.

### DETAILED DESCRIPTION OF THE PREFERRED EMBODIMENTS

Major embodiments of this invention will be explained below, without limiting the scope of this invention.

Note that all numerical ranges given in this patent specification, using “to” preceded and succeeded by numerals, are used to represent the ranges including these numerals respectively as the lower and upper limit values.

The water-based cosmetic of this invention contains 2 to 18% by mass of a water-insoluble water absorbent polymer, 15 to 95% by mass of water, and 0.5 to 45% by mass of a powder, the water-based cosmetic having a penetration of 0.1 N or larger, where the penetration being hardness measured by using a pressure sensing shaft with a 10-mm-diameter spherical end, at a penetration speed of 6 cm/min, an insertion depth of 10 mm, and a temperature of 25° C.

With such structure, this invention becomes able to provide a water-based cosmetic that excels in adhesion, quick-drying property, spreadability without causing uneven application, and easiness of fine control of color density. The aforementioned objects were supposedly attained, particularly by employing the water-insoluble water absorbent polymer having not been used in the field of cosmetic, and by properly adjusting the content ratio to the powder, and the content ratio to water.

The water-based cosmetic of this invention will be explained below.

### [Water-Insoluble Water Absorbent Polymer]

The water-based cosmetic of this invention contains a water-insoluble water absorbent polymer.

Now the term “water-insoluble” in the “water-insoluble water absorbent polymer” means that the polymer is substantially insoluble to water. “Substantially insoluble to water” in the context of this patent specification means that the solubility to water (25° C.) is 5% by mass or smaller, which is more preferably 3% by mass or smaller, and particularly 1% by mass or smaller. The lower limit value, although not specifically limited, is practically 1 ppm (mass basis) or above.

On the other hand, “water absorbent (absorption)” in the “water absorbent polymer” means water absorption capacity represented by a swelling ratio of preferably 1 g/g or larger, more preferably 2 g/g or larger, even more preferably 5 g/g or larger, yet more preferably 10 g/g or larger, and furthermore preferably 15 g/g or larger. The adsorbent is preferably able to naturally absorb aqueous fluid, which is more specifically water, and particularly purified water. The upper limit value, although not specifically limited, is practically 2000 g/g or below, more practically 200 g/g or below, even more practically 150 g/g or below, yet more practically 100 g/g or below, furthermore practically 50 g/g or below, and still furthermore practically 40 g/g or below. Such definition of water absorption is on the basis of measurement at 25° C. and 101,325 Pa as described below, unless otherwise specifically noted.

The swelling ratio is measured by the method below.

A water-insoluble water absorbent polymer (dry weight A) and purified water are mixed by using a disper for 5 minutes, and allowed to stand at 25° C. for 12 hours. After checking there is unabsorbed purified water remaining in the upper area of the container after standing, the purified water in the upper area is suck off with a syringe, and weight B of water-insoluble water absorbent polymer that fully absorbed water is measured. Using the thus obtained weight B, the swelling ratio is calculated by the equation below:

Swelling ratio (g/g)=[Wet weight B (g)−Dry weight A (g)]/Dry weight A (g)

Note, however, in a case where there is no unabsorbed purified water found in the upper area of the container, and the water has been entirely absorbed, re-adjustment is necessary since whether the water-insoluble water absorbent polymer has been saturated or not cannot be deteLudned. For the re-adjustment, the water-insoluble water absorbent polymer having already absorbed water will not be re-used.

The water-insoluble water absorbent polymer used for this invention, when available in the form of particle, preferably has a particle size (D₅₀) of 100 μm or smaller, which is more preferably 50 μm or smaller. The lower limit value, although not specifically limited, is practically 1 μm or larger. The water-insoluble water absorbent polymer used in this invention is preferably provided in the form of spherical powder.

The water-insoluble water absorbent polymer preferably has a swollen particle size (D₅₀) of 1 μm or larger, which is more preferably 2 μm or larger, even more preferably 5 μm or larger, and yet more preferably 10 μm or larger. The upper limit value is preferably 1000 μm or below, more preferably 500 μm or below, even more preferably 200 μm or below, furthermore preferably 150 μm or below, again furthermore preferably 100 μm or below, and still furthermore preferably 50 μm or below.

Note now that, the particle size and the swollen particle size of the water-insoluble water absorbent polymer in this invention are given by D₅₀ values deteiiuined by laser diffractometry/scatterometry. An apparatus used here is a laser diffraction/scattering particle size distribution analyzer LA-950 (from HORIBA, Ltd.). The swollen particle size means a particle size of water-insoluble water absorbent polymer after being allowed to absorb purified water at 25° C. for 12 hours, as has been described above in conjunction with swelling ratio.

Note these absorption characteristics are defined in conjunction with purified water under conditions including standard temperature (25° C.) and pressure (760 mmHg, or 101,325 Pa).

The water-insoluble water absorbent polymer usable in this invention may be any of those if being water-insoluble and capable of absorbing water, and may be preferably provided in the form of microparticle, exemplified by crosslinked product of polyacrylate salt, or cellulose derivative enumerated below, optionally combined with other materials.

The water-insoluble water absorbent polymer is exemplified by crosslinked product of polyacrylate salt produced by polymerizing a mixture of acrylic acid and sodium hydroxide or potassium hydroxide in the presence of an initiator, to thereby form chloride of polyacrylic acid (sodium polyacrylate, potassium polyacrylate, etc.); and products further combined with other materials, such as polyacrylamide copolymer, ethylene maleic anhydride copolymer, crosslinked carboxymethyl cellulose, polyvinyl alcohol copolymer, crosslinked polyethylene oxide, and starch polyacrylonitrile graft copolymer. These products may have controlled swelling ratio depending on the degree of polymerization. Examples include products sold by Avecia, Inc. under the trade names of Octacare X100, X110 and RM100; products sold by SNF under the trade names of Flocare GB300 and Flosorb 500; products sold by BASF SA under the trade names of Luquasorb 1003, Luquasorb 1010, Luquasorb 1280 and Luquasorb 1100; products sold by Grain Processing Coiporation under the trade names of Water Lock G400 and G430 (INCI name: acrylamide/sodium acrylate copolymer); AQUA KEEP 10SH-NF and AQUA KEEP 10SH-NFC supplied from Sumitomo Seika Chemicals Co., Ltd.; crosslinked sodium polyacrylate such as ARON NT-Z (INCI name: sodium acrylate crosspolymer-2) supplied from Nikko Chemicals Co., Ltd.; acryl polymer (homopolymer or copolymer)-grafted starch, which is more specifically sodium polyacrylate-grafted starch such as those sold by Sanyo Chemical Industries, Ltd. under the trade names of Sanfresh ST-100C, ST100MC and IM-300MC (INCI name: sodium polyacrylate starch); and acryl polymer (homopolymer or copolymer)-grafted hydrolyzed starch, which is more specifically hydrolyzed starch grafted with acryloacrylamide/sodium acrylate copolymer, such as those sold by Grain Processing corporation under the trade names of Water Lock A-240, A-180, B-204, D-223, A-100, C-200 and D-223 (INCI name: starch/acrylamide/sodium acrylate copolymer).

Preferred commercialized products are exemplified by crosslinked product of polyacrylate salt: ARON NT-Z (trade name) (From Nikko Chemicals Co., Ltd.); and AQUA KEEP 10SH-NFC (trade name) (Sumitomo Seika Chemicals Co., Ltd.) (both having INCI name: sodium acrylate cross polymer-2, indication name: acrylates crosspolymer-2-Na).

For the water-insoluble water absorbent polymer, also suitably used are those made of spherical particles of polysaccharide having carboxy groups only at the 6-positions and the reducing terminal. The polysaccharide having the carboxy groups may reside within a range recessed from the surface of particle. The polysaccharide is exemplified by cellulose, amylose, amylopectin, pullulan, chitin, chitosan, alginic acid, dextrin, dextran, glycogen, agarose, hyaluronic acid, glucomannan and carrageenan. Among them, cellulose and derivatives thereof are preferred. The water-insoluble water absorbent polymer may also be prepared from commercially available cellulose bead by way of TEMPO oxidation.

For moisturizing particle of water-insoluble water absorbent polymer, made of the aforementioned polysaccharides, descriptions of JP-2018-002879A and JP-2019-116521A may be referred to, specific examples disclosed therein are incorporated by reference into this patent specification.

The water-insoluble water absorbent polymer demonstrates adhesiveness, as a result of leaching of the water having been absorbed therein, when applied on skin or the like, and admixed with the powder described in the paragraphs later. The formulation is preferably designed so that such behavior is suitably demonstrated in the system.

The water-insoluble water absorbent polymer preferably resides in the water-based cosmetic, while being not fully saturated with water, so that percentage of content (% by mass) of an aqueous medium, to a water absorption capacity given by a product of content (% by mass) of the water-insoluble water absorbent polymer and swelling ratio (g/g), will be smaller than the swelling ratio of the water-insoluble water absorbent polymer. That is, the content (% by mass) of the aqueous medium in the water-based cosmetic is calculated by [Content of water-insoluble water absorbent polymer (% by mass)×Swelling ratio of polymer (g/g)], which is preferably less than 130% (mass basis, the same shall apply hereinafter), more preferably less than 100%, even more preferably less than 80%, yet more preferably less than 70%, and furthermore preferably less than 60%. The lower limit value is preferably 5% or above, more preferably 10% or above, even more preferably 15% or above, and yet more preferably 20% or above. The aqueous medium is now defined to encompass water, and a water-soluble liquid component such as polyhydric alcohol, lower alcohol, water-soluble dye, water-soluble coating agent and viscosity modifier.

The content of the water-insoluble water absorbent polymer in the water-based cosmetic is 2% by mass or more, more preferably 2.5% by mass or more, even more preferably 3% by mass or more, yet more preferably 4% by mass or more, and furthermore preferably 4.5% by mass or more. The upper limit value is 18% by mass or below, more preferably 16% by mass or below, even more preferably 12% by mass or below, yet more preferably 10% by mass or below, and furthermore preferably 8% by mass or below.

The water-based cosmetic of this invention may contain only one kind of, or two or more kinds of water-insoluble water absorbent polymer. When two or more kinds are contained, the total content preferably falls in the aforementioned ranges.

### [Powder]

The water-based cosmetic of this invention contains a powder. Any powder that are usually used for cosmetic may be used without special limitation. Also geometry of the powder is not specifically limited, allowing shapes including sphere, plate and needle, existing faults of fume and microparticle, particle size of pigment grade, and structures including porous or non-porous ones.

The powder is specifically exemplified by coloring pigment, pearl and extender pigment. These powders function to add makeup effects such as coloring and brightening of the skin, making the cosmetic shapable or bulky, and improving the touch of use.

Specific examples include extender pigments such as mica, synthetic mica, sericite, talc, kaolin, silicon carbide, barium sulfate, bentonite, smectite, aluminum oxide, silica, magnesium oxide, zirconium oxide, magnesium carbonate, calcium carbonate, chromium oxide, aluminum hydroxide and magnesium hydroxide; UV scattering agents such as microparticular titanium oxide and microparticular zinc oxide; organic powders such as nylon powder, polymethyl methacrylate powder, acrylonitrile-methacrylic acid copolymer powder, vinylidene chloride-methacrylic acid copolymer powder, polyethylene powder, polystyrene powder, organopolysiloxane elastomer powder, polymethylsilsesquioxane powder, urethane powder, wool powder, silk powder, cellulose powder and N-acyllysine powder; composite powders such as microparticular titanium oxide-coated mica titanium, microparticular titanium oxide-coated nylon, barium sulfate-coated mica titanium, titanium oxide-containing silica and zinc oxide-containing silica; and metallic soaps such as magnesium stearate, zinc myristate, aluminum stearate and calcium stearate.

The coloring pigment is exemplified by inorganic coloring pigments such as red iron oxide, yellow iron oxide, black iron oxide, cobalt oxide, chromium oxide, ultramarine, Prussian blue, titanium oxide and zinc oxide; organic coloring pigments such as Red No.228, Red No.226, Blue No.404, Red No.202, Yellow No.4 and aluminum lake; pearl pigments such as titanium mica, microparticular titanium oxide-coated mica titanium, barium sulfate-coated mica titanium, argentine, bismuth oxychloride and aluminum flake; and natural dyes such as carmine and safflower.

The water-based cosmetic of this invention preferably contains, as the powder, a hydrophobic powder. Such hydrophobic powder is less likely to allow the water-insoluble water absorbent polymer to adhere thereon, thus adding powdery touch of use to the cosmetic. The hydrophobic powder may only be a powder whose surface demonstrates hydrophobicity, and is exemplified by an intrinsically hydrophobic powder, a hydrophilic powder treated with a hydrophobicizing agent, and in pursuit of further enhanced hydrophobicity, an intrinsically hydrophobic powder further treated with a hydrophobicizing agent. The surface treatment agent for hydrophobicizing is exemplified by higher fatty acid, metal soap, oil and fat, wax, silicone compound, fluorine-containing compound, surfactant, and dextrin fatty acid ester. The hydrophobicized powder is exemplified by dimethicone-treated powder marketed by Kiyoshi Kasei, Inc.; FHS (perfluorohexylethyltriethoxysilane)-treated powder and OTS (triethoxycaprylylsilane)-treated powder marketed by Daito Kasei Kogyo Co., Ltd.; and silicone-treated powder marketed by Topy Industries, Ltd. Now, “hydrophobic” means that the powder does not disperse in water even if stirred therein.

In the water-based cosmetic of this invention, content of the powder is 0.5% by mass or more, preferably 2% by mass or more, more preferably 4% by mass or more, even more preferably 6% by mass or more, yet more preferably 8% by mass or more, and furthermore preferably 10% by mass or more. The upper limit of the powder content is 45% by mass or below, preferably 40% by mass or below, more preferably 37% by mass or below, and even more preferably 35% by mass or below.

The water-based cosmetic of this invention may contain only one kind of, or two or more kinds of powder. When two or more kinds are contained, the total content preferably falls in the aforementioned ranges.

With the content of powder controlled within the aforementioned ranges, water having been absorbed in the water-insoluble water absorbent polymer can leach out when the cosmetic is applied on skin or the like, and can admix with the powder, thus supposedly demonstrating proper adhesion.

Again in the water-based cosmetic of this invention, content of the hydrophobic powder is preferably 0.01% by mass or more, more preferably 0.5% by mass or more, even more preferably 3% by mass or more, yet more preferably 5% by mass or more, and furthermore preferably 10% by mass or more. The upper limit of the powder content is preferably 45% by mass or below, more preferably 40% by mass or below, and even more preferably 35% by mass or below.

The water-based cosmetic of this invention may contain only one kind of, or two or more kinds of hydrophobic powder. When two or more kinds are contained, the total content preferably falls in the aforementioned ranges.

[Water and Aqueous Medium Other than Water]

The water-based cosmetic of this invention contains water. The water includes purified water, distilled water, ion exchanged water, pure water and ultrapure water, wherein purified water is preferred.

Content of water in the water-based cosmetic, in this invention, is 15% by mass or more, preferably 20% by mass or more, more preferably 25% by mass or more, and even more preferably 30% by mass or more. The upper limit is 95% by mass or below, preferably 90% by mass or below, more preferably 85% by mass or below, even more preferably 80% by mass or below, and yet more preferably 70% by mass or below.

In the water-based cosmetic of this invention, also a mixture of water and an aqueous medium other than water may be used. The aqueous medium is exemplified by alcohol, wherein polyhydric alcohol is particularly preferred in this invention. The alcohol is exemplified by BG (1,3-butylene glycol), penthylene glycol, dipropylene glycol, glycerin and ethanol.

Content of the aqueous medium, when contained in the water-based cosmetic of this invention, is preferably 1% by mass or more of the water-based cosmetic, which is more preferably 3% by mass or more, even more preferably 4% by mass or more, yet more preferably 5% by mass or more, and if high content is desired, the content is preferably 35% by mass or more, more preferably 45% by mass or more, and even more preferably 55% by mass or more. Meanwhile, the content of the aqueous medium is preferably 98% by mass or less, more preferably 95% by mass or less, even more preferably 90% by mass or less, and if low content is desired, the content is preferably 40% by mass or less, more preferably 37% by mass or less, even more preferably 36% by mass or less, and yet more preferably 35% by mass or less.

The water-based cosmetic of this invention may contain only one kind of, or two or more kinds of aqueous medium. When two or more kinds are contained, the total content preferably falls in the aforementioned ranges.

### [Water-Soluble Dye]

The water-based cosmetic of this invention may contain a water-soluble dye.

The water-soluble dye usable in this invention is exemplified by Red No.3, Red No.104,Red No.106, Red No.227, Red No.230, Red No.401, Red No.505, Orange No.205, Yellow No.4, Yellow No.5, Yellow No.202, Yellow No.203, Green No.3 and Blue No.1.

Content of the water-soluble dye, when used, in the water-based cosmetic is 0.05% by mass or more, and more preferably 0.1% by mass or more, although not specifically limited. The upper limit value is practically 5% by mass or below.

The water-based cosmetic of this invention may contain only one kind of, or two or more kinds of water-soluble dye. When two or more kinds are contained, the total content preferably falls in the aforementioned ranges.

### [Others]

The water-based cosmetic of this invention may contain an oil.

The oil may be any of those having been widely used for cosmetic, without special limitation, and is exemplified by wax, paraffin, higher fatty acid, oils and fats, hydrocarbon oil, ester oil, silicone oil and higher alcohol, among which preferred are ester oil, hydrocarbon oil and silicone oil which exist in the form of liquid or paste at 25° C.

Content of the oil is preferably 0 to 15% by mass. With the content of oil controlled to 15% by mass or less, the quick-drying property will be more effectively improved, and uneven application may more effectively be suppressed. The content of oil is preferably 0.1 to 10% by mass, and more preferably 1 to 5% by mass.

The water-based cosmetic of this invention may contain only one kind of, or two or more kinds of oil. When two or more kinds are contained, the total content preferably falls in the aforementioned ranges.

The water-based cosmetic of this invention may also contain high-FILE surfactant, coating agent, viscosity modifier, thickener and so forth. With these ingredients contained therein, the adhesion will more likely to improve. Besides these ingredients, the water-based cosmetic of this invention may still further contain any optional ingredients having been used for usual water-based cosmetic. More specifically, proper amounts of antioxidant, neutralizing agent, UV absorber, chelating agent, beauty ingredient, fragrance or pH adjuster may be contained without reducing the effects of invention.

The water-based cosmetic of this invention is preferably, but not exclusively, substantially free of low-HLB surfactant, or oil that exists in the form of solid at 25° C. (wax, paraffin, higher fatty acid, etc.). “Substantially free of . . . ” means that the content is 1% by mass or less of the water-based cosmetic of this invention, which is more preferably 0.1% by mass or less, even more preferably 0.01% by mass or less, yet more preferably 0.001% by mass or less, and furthermore preferably 0% by mass.

The water-based cosmetic of this invention is blended so that the water-insoluble water absorbent polymer, water, powder and optional other ingredients will total 100% by mass.

The water-based cosmetic of this invention lacks fluidity at room temperature (25° C.), and is therefore different from those in the form of liquid of gel. In other words, product foLm of the water-based cosmetic this invention is preferably solid, which may be represented by penetration. The penetration is more specifically given by hardness which may be measured by using, for example, a hardness measured by using a rheometer (from Rheotech Co., Ltd.) whose pressure sensing shaft has a spherical end of 10 mm in diameter, under conditions involving a penetration speed of 6 cm/min, an insertion depth of 10 mm, and a temperature of 25° C. The water-based cosmetic of this invention preferably has a hardness of 0.1 N or larger, which is preferably 0.3 N or larger, and more preferably 1 N or larger. The cosmetic, when given in the form of gel, typically has a hardness smaller than 0.1 N. The upper limit value is preferably, but not restrictively, 25 N or smaller for example, and more preferably 15 N or smaller.

The water-based cosmetic of this invention is applicable to broad lineups of items, which are exemplified by makeup cosmetics (eyebrow pencil, eyeliner, eye shadow, lipstick, blush and foundation), hair color cosmetic (temporary hair color) and so forth.

The water-based cosmetics according to the preferred embodiments of this invention may yield advantages as follows. That is, blending of a proper amount of water-insoluble water absorbent polymer allows water to leach under pressure when the cosmetic is applied, so as to create cool and refreshing sensation, as well as good spreadability and adhesion. On the other hand, unsaturated water-insoluble water absorbent polymer absorbs water leached out during the application, to demonstrate quick-drying property. Moreover, properly specified amounts of the powder, the water-insoluble water absorbent polymer and water are blended to enable fine control of color density, and suitably balanced for the operation. Use of a proper amount of powder (hydrophobic powder in a particularly preferred embodiment) also makes it easier to finely control color density during application.

### EXAMPLES

This invention will further be detailed below referring to Examples, with which this invention will not be understood limitedly. “%” means “% by mass”, unless otherwise specifically noted.

Cosmetics whose ingredient compositions are summarized in Tables 1 to 8 were prepared as described below. The individual ingredients in Table 1 are given on the mass basis.

Referring now to Example 1, surfactants (polyoxyethylene alkyl ether, polyoxyethylene glyceryl fatty acid ester) and an aqueous medium (surfactant, if solid, was dissolved under heating) were mixed in a disper, or by stirring with a spatula. The powder ingredient was then fed, and the content was mixed by using a kneader or mixer to prepare a cosmetic.

Also cosmetics other than Example 1 were prepared in the same way except that the ingredient compositions summarized in Tables below were used.

The water-based cosmetic prepared in Example 1 was subjected to penetration test by using a rheometer, as specified previously. The hardness was confirmed to exceed 0.1 N. Also the water-based cosmetics of the individual Examples were subjected to the penetration test in the same way, to confirm that the hardness exceeded 0.1 N.

The thus prepared cosmetics were tested as follows. That is, each cosmetic was applied so as to draw a line of 5 cm long on skin by using a chip-type applicator, and then evaluated regarding the items below. Results were summarized in Tables 1 to 5 below. The cosmetics ranked C for any item of evaluation are poor and inferior in marketability.

### <Quick-Drying Property>

Evaluation method: After the elapse of a certain period of time, the line was touched by a finger to check the quick-drying property. An average value based on five times of trial, participated by a single panelist, was employed as an evaluation value.

### Evaluation Criteria

AA: dried within 30 seconds;

A: dried after 30 seconds and within 1 minute;

B: dried after 1 minute and within 2 minutes; and

C: not dried even after 2 minutes.

### <Adhesion>

Evaluation method: How many times of application was necessary for fixation of the drawn line was checked. An average value based on five times of trial, participated by a single panelist, was employed as an evaluation value.

### Evaluation Criteria:

AA: once;

A: more than once and within 4 times;

B: more than 4 times and within 9 times; and

C: more than 9 times, or not fixed.

### <Unevenness-Free>

Evaluation method: Each cosmetic was applied five times, and whether there were unevenness in the drawn line was checked. An average value based on five times of trial, participated by a single panelist, was employed as an evaluation value.

### Evaluation Criteria:

A: unevenness not found;

B: unevenness found, but correctable with an applicator; and

C: unevenness found, and not correctable.

### <Easiness of Fine Control of Color Density>

Evaluation method: Each cosmetic was applied five times, and easiness of fine control of color density was evaluated by sensory evaluation participated by 10 panelists. The results were averaged, and evaluated according to the ranks below.

### Evaluation Criteria:

A: eight or more panelists answered it was easy;

B: four or more and seven or less panelists answered it was easy; and

C: three or less panelists answered it was easy.

TABLE 1

| | | Eyebrow pencil | | | | | | |
|---|---|---|---|---|---|---|---|---|
| | Item | Example | Example | Example | Example | Example | Example | Example |
| | Ingredient | 1 | 2 | 3 | 4 | 5 | 6 | 7 |
| Alcohol | BG | 15 | 15 | 10 | 10 | 15 | 15 | 15 |
| Viscosity | Xanthane gum | | | | | | | 0.2 |
| modifier | PVP | 0.5 | 0.5 | | | | | 0.5 |
| Coloring | Carbon black | 0.1 | 0.3 | 0.2 | 0.2 | 0.3 | 0.1 | 0.2 |
| pigment | Iron oxide | 2 | 3 | 3 | 3 | 3 | 3 | 2 |
| | Titanium oxide | | | | | | | 2.5 |
| Water- | Yellow No. 4 | 0.1 | 0.1 | 0.1 | 0.1 | 0.1 | | |
| soluble dye | Red No. 227 | | | | | | | |
| | Blue No. 1 | | | | | | | |
| Extender | Dimethicone-treated | 18 | 16 | 15 | 15 | 33 | 15 | 5 |
| pigment | synthetic phlogopite | | | | | | | |
| | Synthetic phlogopite | | | | | | | 2 |
| Water-insoluble | Acrylates crosspolymer-2-Na *1 | 6 | 6 | 6 | 10 | 8 | 6 | 5.5 |
| water absorbent | Acrylates crosspolymer-2-Na *2 | | | | | | | |
| polymer | | | | | | | | |
| Water | Purified water | 57.6 | 58.4 | 65 | 61 | 39.9 | 60.2 | 66.4 |
| Others | Preservative | 0.5 | 0.5 | 0.5 | 0.5 | 0.5 | 0.5 | 0.5 |
| | Polyoxyethylene alkyl ether | 0.1 | 0.1 | 0.1 | 0.1 | 0.1 | 0.1 | 0.1 |
| | Polyoxyethylene glyceryl | 0.1 | 0.1 | 0.1 | 0.1 | 0.1 | 0.1 | 0.1 |
| | fatty acid ester | | | | | | | |
| Total | | 100 | 100 | 100 | 100 | 100 | 100 | 100 |
| Water absorption rate | | 48% | 49% | 50% | 28% | 27% | 50% | 59% |
| Evaluation | Quick-drying property | AA | AA | AA | AA | AA | AA | AA |
| | Adhesion | AA | AA | AA | AA | AA | AA | AA |
| | Unevenness-free (spreadability) | A | A | A | A | A | A | A |
| | Easiness of fine control | A | A | A | A | A | A | A |
| | of color density | | | | | | | |

| | | Eyebrow pencil | | | | | |
|---|---|---|---|---|---|---|---|
| | Item | Example | Example | Example | Example | Example | Example |
| | Ingredient | 8 | 9 | 10 | 11 | 12 | 13 |
| Alcohol | BG | 15 | 10 | 5 | 5 | 30 | 20 |
| Viscosity | Xanthane gum | | | | | | |
| modifier | PVP | | 0.5 | | | 0.5 | |
| Coloring | Carbon black | 10 | 0.9 | 0.2 | | 0.3 | 1 |
| pigment | Iron oxide | 6 | 2 | 2.5 | | 3 | 1 |
| | Titanium oxide | 6 | | | | | |
| Water- | Yellow No. 4 | 0.1 | | | 0.2 | 0.1 | 0.3 |
| soluble dye | Red No. 227 | | | | 0.2 | | 0.3 |
| | Blue No. 1 | | 0.1 | | 0.1 | | 0.2 |
| Extender | Dimethicone-treated | 20 | 16.5 | 30 | 0.5 | 39.4 | 40 |
| pigment | synthetic phlogopite | | | | | | |
| | Synthetic phlogopite | | | | | | |
| Water-insoluble | Acrylates crosspolymer-2-Na *1 | 8 | | 0.5 | 3 | 6 | 2 |
| water absorbent | Acrylates crosspolymer-2-Na *2 | | 3.5 | 2 | | | |
| polymer | | | | | | | |
| Water | Purified water | 34.2 | 65.4 | 59 | 90.5 | 20 | 34.5 |
| Others | Preservative | 0.5 | 0.5 | 0.5 | 0.5 | 0.5 | 0.5 |
| | Polyoxyethylene alkyl ether | 0.1 | 0.5 | 0.3 | | 0.1 | 0.2 |
| | Polyoxyethylene glyceryl | 0.1 | 0.1 | | | 0.1 | |
| | fatty acid ester | | | | | | |
| Total | | 100 | 100 | 100 | 100 | 100 | 100 |
| Water absorption rate | | 25% | 18% | 25% | 127% | 33% | 109% |
| Evaluation | Quick-drying property | AA | AA | AA | A | A | B |
| | Adhesion | AA | AA | AA | A | B | B |
| | Unevenness-free (spreadability) | A | B | A | A | B | A |
| | Easiness of fine control | A | A | A | A | B | A |
| | of color density | | | | | | |

TABLE 2

| | | Eyebrow pencil | | | | | | | |
|---|---|---|---|---|---|---|---|---|---|
| | | | | Compara- | Compara- | Compara- | Compara- | Compara- | Compara- |
| | | | | tive | tive | tive | tive | tive | tive |
| | Item | Example | Example | Example | Example | Example | Example | Example | Example |
| | Ingredient | 14 | 15 | 1 | 2 | 3 | 4 | 5 | 6 |
| Alcohol | BG | 35 | 1 | 0.5 | 63.55 | 15 | 15 | 15 | 1 |
| Viscosity | Xanthane gum | 0.5 | | | | | | | |
| modifier | PVP | 1 | | 0.5 | 0.5 | 0.5 | 0.5 | 0.5 | 0.5 |
| Coloring | Carbon black | 0.2 | | 0.1 | 0.2 | 0.1 | 0.1 | 0.1 | 0.1 |
| pigment | Iron oxide | 2 | | | 3 | 3 | 3 | 3 | 2.5 |
| | Titanium oxide | | | | | | | | |
| Water- | Yellow No. 4 | 0.8 | 0.3 | | 0.1 | 0.1 | 0.1 | 0.1 | 0.1 |
| soluble dye | Red No. 227 | 0.5 | 0.3 | | | | | | |
| | Blue No. 1 | 0.5 | 0.1 | | | | | | |
| Extender | Dimethicone-treated | 10 | 5 | 0.5 | 16 | 16 | 16 | 16 | 50 |
| pigment | synthetic phlogopite | | | | | | | | |
| | Synthetic phlogopite | | | | | | | | |
| Water-insoluble | Acrylates crosspolymer-2-Na *1 | 17.5 | 10 | 2 | 5.5 | 20 | 0.2 | | 5 |
| water absorbent | Acrylates crosspolymer-2-Na *2 | | | | | | | | |
| polymer | | | | | | | | | |
| Water | Purified water | 31.2 | 82.8 | 95.7 | 10.45 | 44.6 | 64.4 | 64.6 | 40.1 |
| Others | Preservative | 0.5 | 0.5 | 0.5 | 0.5 | 0.5 | 0.5 | 0.5 | 0.5 |
| | Polyoxyethylene alkyl ether | 0.3 | | 0.1 | 0.1 | 0.1 | 0.1 | 0.1 | 0.1 |
| | Polyoxyethylene glyceryl | | | 0.1 | 0.1 | 0.1 | 0.1 | 0.1 | 0.1 |
| | fatty acid ester | | | | | | | | |
| Total | | 100 | 100 | 100 | 100 | 100 | 100 | 100 | 100 |
| Water absorption rate | | 15% | 34% | 192% | 54% | 12% | 1588% | | 33% |
| Evaluation | Quick-drying property | A | A | B | A | C | C | C | AA |
| | Adhesion | AA | AA | A | B | AA | A | C | C |
| | Unevenness-free (spreadability) | A | A | C | C | A | B | B | A |
| | Easiness of fine control | A | A | C | C | C | C | C | C |
| | of color density | | | | | | | | |

TABLE 3
Item: Eyebrow pencil

| | | Exam- | Exam- | Exam- | Exam- | Exam- | Exam- | Exam- | Exam- | Exam- | Exam- |
|---|---|---|---|---|---|---|---|---|---|---|---|
| | | ple | ple | ple | ple | ple | ple | ple | ple | ple | ple |
| | Ingredient | 16 | 17 | 18 | 19 | 20 | 21 | 22 | 23 | 24 | 25 |
| Alcohols | BG | | | | 15 | 15 | 15 | 15 | 15 | 15 | 10 |
| | Pentylene glycol | 15 | | | | | | | | | |
| | Glycerin | | 15 | | | | | | | | |
| | Ethanol | | | 15 | | | | | | | |
| Viscosity | PVP | 0.5 | 0.5 | 0.5 | 0.5 | 0.5 | 0.5 | 0.5 | 0.5 | 0.5 | |
| modifier | | | | | | | | | | | |
| Coloring | Carbon black | 0.3 | 0.3 | 0.3 | 0.3 | 0.3 | 0.3 | 0.3 | 0.3 | 0.3 | 0.2 |
| pigment | Iron oxide | 3 | 3 | 3 | 3 | 3 | 3 | 3 | 3 | 3 | 2.5 |
| Water- | Red No. 227 | | | | | | | | | | |
| soluble dye | Yellow No. 4 | 0.1 | 0.1 | 0.1 | 0.1 | 0.1 | 0.1 | 0.1 | 0.1 | 0.1 | |
| | Blue No. 1 | | | | | | | | | | |
| Extender | Dimethicone-treated | 16 | 16 | 16 | | | | | | | 13 |
| pigment | synthetic phlogopite | | | | | | | | | | |
| | Dimethicone-treated sericite | | | | 16 | | | | | | |
| | Dimethicone-treated talc | | | | | 16 | | | | | |
| | Dimethicone-treated silica | | | | | | 16 | | | | |
| | Synthetic phlogopite | | | | | | | 16 | | | |
| | Talc | | | | | | | | 16 | | |
| | Silica | | | | | | | | | 16 | |
| Water-insoluble | Acrylates crosspolymer-2-Na *1 | 6 | 6 | 6 | 6 | 6 | 6 | 6 | 6 | 6 | 5 |
| water absorbent | | | | | | | | | | | |
| polymer | | | | | | | | | | | |
| Water | Purified water | 58.4 | 58.4 | 58.4 | 58.4 | 58.4 | 58.4 | 58.4 | 58.4 | 58.4 | 63.7 |
| Others | Preservative | 0.5 | 0.5 | 0.5 | 0.5 | 0.5 | 0.5 | 0.5 | 0.5 | 0.5 | 0.5 |
| | Polyoxyethylene alkyl ether | 0.1 | 0.1 | 0.1 | 0.1 | 0.1 | 0.1 | 0.1 | 0.1 | 0.1 | 0.1 |
| | Polyoxyethylene glyceryl | 0.1 | 0.1 | 0.1 | 0.1 | 0.1 | 0.1 | 0.1 | 0.1 | 0.1 | |
| | fatty acid ester | | | | | | | | | | |
| | Neopentyl glycol diethylhexanoate | | | | | | | | | | 5 |
| Total | | 100 | 100 | 100 | 100 | 100 | 100 | 100 | 100 | 100 | 100 |
| Water absorption rate | | 49% | 49% | 49% | 49% | 49% | 49% | 49% | 49% | 49% | 59% |
| Evaluation | Quick-drying property | AA | AA | AA | AA | AA | AA | AA | AA | AA | A |
| | Adhesion | AA | AA | AA | AA | AA | AA | AA | AA | AA | AA |
| | Unevenness-free (spreadability) | A | A | A | A | A | A | A | A | A | A |
| | Easiness of fine control | A | A | A | A | A | A | B | B | B | B |
| | of color density | | | | | | | | | | |

TABLE 4
Example 26 Eyeliner

| | |
|---|---|
| BG | 15% |
| PVP | 0.5% |
| Alkyl acrylate derivative coating agent | 2.5% |
| Carbon black | 10% |
| Dimethicone-treated sericite | 16% |
| Acrylates crosspolymer-2-Na *1 | 6% |
| Purified water | 49% |
| Preservative | 0.5% |
| Polyoxyethylene alkyl ether | 0.1% |
| Polyoxyethylene glyceryl fatty acid ester | 0.1% |
| Quick-drying property | AA |
| Adhesion | AA |
| Unevenness-free (spreadability) | A |
| Easiness of fine control of color density | A |

Example 27 Eyeliner

| | |
|---|---|
| BG | 15% |
| Ethanol | 3% |
| PVP | 0.5% |
| Carbon black | 10% |
| Acrylic derivative coating agent *3 | 2% |
| Dimethicone-treated synthetic phlogopite | 16% |
| Acrylates crosspolymer-2-Na *1 | 6% |
| Purified water | 46.8% |
| Preservative | 0.5% |
| Polyoxyethylene alkyl ether | 0.1% |
| Polyoxyethylene glyceryl fatty acid ester | 0.1% |
| Quick-drying property | AA |
| Adhesion | AA |
| Unevenness-free (spreadability) | A |
| Easiness of fine control of color density | A |

TABLE 5
Example 28 Eyeliner

| | |
|---|---|
| BG | 14% |
| Carbon black | 1% |
| Blue No. 1 | 0.1% |
| Dimethicone-treated synthetic phlogopite | 10% |
| Acrylates crosspolymer-2-Na *1 | 7% |
| Purified water | 67.2% |
| Preservative | 0.5% |
| Polyoxyethylene alkyl ether | 0.1% |
| Polyoxyethylene glyceryl fatty acid ester | 0.1% |
| Quick-drying property | AA |
| Adhesion | AA |
| Unevenness-free (spreadability) | A |
| Easiness of fine control of color density | A |

| Example 29 Eyebrow pencil | |
|---|---|
| BG | 15% |
| Carbon black | 4% |
| Prussian blue | 0.1% |
| Blue No. 1 | 0.1% |
| Dimethicone-treated synthetic phlogopite | 15% |
| Acrylates crosspolymer-2-Na *1 | 5% |
| Purified water | 60% |
| Preservative | 0.5% |
| Polyoxyethylene alkyl ether | 0.1% |
| Polyoxyethylene glyceryl fatty acid ester | 0.1% |
| Quick-drying property | AA |
| Adhesion | AA |
| Unevenness-free (spreadability) | A |
| Easiness of fine control of color density | A |

TABLE 6
Example 30 Lip cosmetic

| | |
|---|---|
| BG | 10% |
| Red No. 227 | 0.3% |
| Yellow No. 4 | 0.3% |
| Blue No. 1 | 0.2% |
| Dimethicone-treated synthetic phlogopite | 5% |
| Acrylates crosspolymer-2-Na *1 | 7% |
| Purified water | 76.7% |
| Preservative | 0.5% |
| Quick-drying property | AA |
| Adhesion | AA |
| Unevenness-free (spreadability) | A |
| Easiness of fine control of color density | A |

Example 31 Blush

| | |
|---|---|
| BG | 5% |
| Red No. 227 | 0.3% |
| Yellow No. 4 | 0.2% |
| Blue No. 1 | 0.1% |
| Dimethicone-treated synthetic phlogopite | 10% |
| Acrylates crosspolymer-2-Na *1 | 7% |
| Purified water | 77.4% |
| Quick-drying property | AA |
| Adhesion | AA |
| Unevenness-free (spreadability) | A |
| Easiness of fine control of color density | A |

TABLE 7
Example 32 Eye shadow

| | |
|---|---|
| BG | 10% |
| Iron oxide | 3% |
| Pearl pigment *4 | 3% |
| Pearl pigment *5 | 3.5% |
| Synthetic phlogopite | 3% |
| Acrylates crosspolymer-2-Na *1 | 4.5% |
| Purified water | 72.5% |
| Preservative | 0.5% |
| Quick-drying property | AA |
| Adhesion | AA |
| Unevenness-free (spreadability) | A |
| Easiness of fine control of color density | B |

Example 33 Eye shadow

| | |
|---|---|
| BG | 10% |
| PVP | 1% |
| Iron oxide | 3% |
| Pearl pigment *5 | 6.5% |
| Dimethicone-treated synthetic phlogopite | 3% |
| Synthetic phlogopite | 3% |
| Acrylates crosspolymer-2-Na *1 | 4.5% |
| Purified water | 68.5% |
| Preservative | 0.5% |
| Quick-drying property | AA |
| Adhesion | AA |
| Unevenness-free (spreadability) | A |
| Easiness of fine control of color density | A |

TABLE 8
Example 34 Temporary hair color

| | |
|---|---|
| BG | 15% |
| Xanthane gum | 1.5% |
| Carbon black | 0.5% |
| Red No. 226 | 0.1% |
| Pearl pigment *4 | 3% |
| Pearl pigment *5 | 3.5% |
| Dimethicone-treated synthetic phlogopite | 2% |
| Acrylates crosspolymer-2-Na *1 | 6.5% |
| Purified water | 65.8% |
| Preservative | 0.5% |
| Polyoxyethylene alkyl ether | 0.1% |
| Polyoxyethylene glyceryl fatty acid ester | 1.5% |
| Quick-drying property | AA |
| Adhesion | AA |
| Unevenness-free (spreadability) | A |
| Easiness of fine control of color density | A |

### <Abbreviations in Tables>

BG: 1,3-butylene glycol
PVP: polyvinylpyrrolidone
*1: ARON NT-Z (Nikko Chemicals Co., Ltd.), swelling ratio ca. 25 g/g, swollen particle size (D₅₀): 37 μm
*2: AQUA KEEP 10SH-NFC (Sumitomo Seika Chemicals Co., Ltd.), swelling ratio 120 g/g, swollen particle size (D₅₀): 170 μm
*3: Acrylic derivative coating agent—alkyl acrylate/amide acrylate derivative coating agent
*4: Titanium oxide-coated synthetic phlogopite “Helios R100S” (Topy Industries, Ltd.)
※5: Titanium oxide-coated synthetic phlogopite “Cosmetica Super White 9000S” (CQV)

The swollen particle size is given by D₅₀ of the particle allowed to absorb purified water for 12 hours, measured by laser diffractometry/scatterometry. An apparatus and a preset value for refractive index are as follows:
Apparatus: Laser diffraction/scattering particle size distribution analyzer LA-950 (from HORIBA, Ltd.); and
Preset value of refractive index: 1.53

Percentage of content (% by mass) of an aqueous medium, to a water absorption capacity given by a product of content (% by mass) of the water-insoluble water absorbent polymer and swelling ratio (g/g) in Example 1 was found to be as follows. The percentage is given as “water absorption rate” in Tables 1 and 2.

Content of water-insoluble water absorbent polymer (6% by mass)×swelling ratio (25 g/g)=150

[Aqueous medium (alcohol, water) (72.6% by mass)/150]×100=48 (%)

In Example 1, the powder consists of a coloring pigment and an extender pigment, and the extender pigment is dimethicone-treated synthetic phlogopite which is a hydrophobic powder.

As is clear from the results, the water-based cosmetics of this invention (Examples) were found to excel in all aspects including quick-drying property, adhesion, unevenness-free nature and easiness of fine control of color density. Such excellent characteristics were also found to be demonstrated not only in eyebrow pencil, but also in a wide variety of applicable items including eyeliner, eye shadow, lip cosmetic, blush and temporary hair color.

In contrast, the cosmetics in Comparative Examples 1 and 2 with too much or too less content of water were found to be inferior in unevenness-free nature and easiness of fine control of color density. The cosmetics in which the water-insoluble water absorbent polymer was too much (Comparative Example 3), too short (Comparative Example 4) and not used (Comparative Example 5) were found to be inferior in quick-drying property and easiness of fine control of color density. In particular, Comparative Example 5 using no water-insoluble polymer was also found to be inferior in adhesion. The cosmetic with too much powder (Comparative Example 6) was found to be inferior in adhesion and easiness of fine control of color density.

### Reference Example 1

Potassium polyacrylate (swollen particle size (D₅₀):15 μm, swelling ratio: 20 g/g) and purified water were mixed in the ratio 5:95 (ratio by mass) under stirring, to obtain a gel. The gel was subjected to the above-specified penetration test by using the rheometer (from Rheotech Co., Ltd.), and was confiLmed to have a hardness of below 0.1 N (25° C.).

### Reference Example 2

Behavior of the powder against water was examined in order to define hydrophilicity and hydrophobicity.

### Experimental Conditions

Into a screw cap tube, placed were 30 g of purified water and 1.5 g of each kind of powder, and the mixture was visually observed before stirred, after stirred and allowed to stand for 10 minutes, and after further allowed to stand for one day.

Three following kinds of powder were used, wherein <1> is untreated; and <2> and <3> are hydrophobicized synthetic fluorophlogopites. Base powder employed for <2> and <3> was PDM-Series synthetic fluorophlogopites from Topy Industries, Ltd.

<1> Synthetic fluorophlogopite PDM-5L (from Topy Industries, Ltd.)

<2> Silicone-treated synthetic fluorophlogopite (treated by Topy Industries, Ltd.)

<3> Fluorine-treated synthetic fluorophlogopite (treated by Daito Kasei Kogyo Co., Ltd.)

### Experimental Results

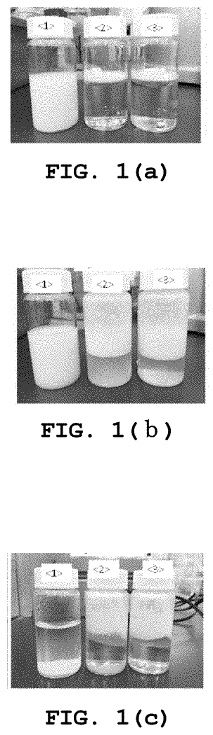

The mixture before stirred is shown in FIG. 1(a), the mixture after stirred and allowed to stand for 10 minutes is shown in FIG. 1(b), and the mixture further allowed to stand for one day is shown in FIG. 1(c).

The synthetic fluorophlogopite <1> was confirmed to disperse in water even before stirred, and to uniformly disperse in water after stirred. The powder was however found to settle after allowed to stand for one day.

The silicone-treated synthetic fluorophlogopite <2> was found to float on the aqueous phase before stirred. The powder also remained floated on the aqueous phase also after stirred and after allowed to stand for one day, rather than dispersed in water, showing a visible boundary between the aqueous phase and powder layer. Also fluorine-treated synthetic fluorophlogopite <3> was found to remain floated on the aqueous phase, rather than dispersed in water, just like the silicone-treated synthetic fluorophlogopite.

### Conclusion

The synthetic fluorophlogopite dispersed in water and then settled but without showing dissolution, proving that it is insoluble. It is also understood to be hydrophilic, since it unifoLmly dispersed in water and was compatible with water, while denying hydrophobicity.

In contrast, the hydrophobicized synthetic fluorophlogopite did not dissolve in water, like the synthetic fluorophlogopite <1>. Moreover, it remained floated again on the aqueous phase even after once stirred, proving its hydrophobicity, or incompatibility with water.

What is claimed is:

1. A water-based cosmetic comprising:
2 to 18% by mass of a water-insoluble water absorbent polymer;
15 to 95% by mass of water; and
0.5 to 45% by mass of a powder,
the water-based cosmetic having a penetration of 0.1 N or larger,
where the penetration being hardness measured by using a pressure sensing shaft with a 10-mm-diameter spherical end, at a penetration speed of 6 cm/min, an insertion depth of 10 mm, and a temperature of 25° C.

2. The water-based cosmetic of claim 1, wherein the water-insoluble water absorbent polymer has a swelling ratio of 5 to 2000 g/g.

3. The water-based cosmetic of claim 1, wherein the water-insoluble water absorbent polymer has a swollen particle size (D₅₀) of 1 μm or larger and 1000 μm or smaller.

4. The water-based cosmetic of claim 1, wherein percentage of content (% by mass) of an aqueous medium, to a water absorption capacity given by a product of content (% by mass) of the water-insoluble water absorbent polymer and swelling ratio (g/g), is 10% or larger and smaller than 130% (mass basis).

5. The water-based cosmetic of claim 1, wherein the water-insoluble water absorbent polymer contains at least one substance selected from crosslinked polyacrylate salt and cellulose derivative.

6. The water-based cosmetic of claim 1, wherein content of the powder is 10 to 40% by mass.

7. The water-based cosmetic of claim 1, wherein the powder contains a hydrophobic powder.

8. The water-based cosmetic of claim 1, wherein the powder contains a coloring pigment.

9. The water-based cosmetic of claim 1, further comprising a water-soluble dye.

10. The water-based cosmetic of claim 1, wherein the penetration of the water-based cosmetic is from 0.3 N to 25 N.

11. The water-based cosmetic of claim 1, wherein the water-insoluble water absorbent polymer contains at least one substance selected from crosslinked polyacrylate salt and cellulose derivative and the powder contains at least one kind of coloring pigment, pearl and extender pigment.

12. The water-based cosmetic of claim 1, further comprising at least one kind of polyhydric alcohol, lower alcohol, water-soluble dye, water-soluble coating agent and viscosity modifier.

13. The water-based cosmetic of claim 2, wherein the water-insoluble water absorbent polymer has a swollen particle size (D₅₀) of 1 μm or larger and 1000 μm or smaller.

14. The water-based cosmetic of claim 2, wherein percentage of content (% by mass) of an aqueous medium, to a water absorption capacity given by a product of content (% by mass) of the water-insoluble water absorbent polymer and swelling ratio (g/g), is 10% or larger and smaller than 130% (mass basis).

15. The water-based cosmetic of claim 2, wherein the water-insoluble water absorbent polymer contains at least one substance selected from crosslinked polyacrylate salt and cellulose derivative.

16. The water-based cosmetic of claim 2, wherein content of the powder is 10 to 40% by mass.

17. The water-based cosmetic of claim 3, wherein percentage of content (% by mass) of an aqueous medium, to a water absoLption capacity given by a product of content (% by mass) of the water-insoluble water absorbent polymer and swelling ratio (g/g), is 10% or larger and smaller than 130% (mass basis).

18. The water-based cosmetic of claim 3, wherein the water-insoluble water absorbent polymer contains at least one substance selected from crosslinked polyacrylate salt and cellulose derivative.

19. The water-based cosmetic of claim 3, wherein content of the powder is 10 to 40% by mass.

20. The water-based cosmetic of claim 4, wherein the water-insoluble water absorbent polymer contains at least one substance selected from crosslinked polyacrylate salt and cellulose derivative and;
wherein content of the powder is 10 to 40% by mass.
